(19) United States
(12) Patent Application Publication
(10) Pub. No.: US 2005/0180956 A1
(43) Pub. Date: Aug. 18, 2005

(54) SSAO enzyme substrates for vasorelaxation, and methods of use thereof

(76) Inventor: Daniel J. Conklin, Louisville, KY (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402, US

(21) Appl. No.: 10/781,318

(22) Filed: Feb. 18, 2004

(51) Int. Cl. A61K 45/00; A61K 31/195; A61K 31/138; A61K 31/137

(52) U.S. Cl. 424/93.7; 514/567; 514/649; 514/651

(57) ABSTRACT

Another embodiment relates to a method of processing a blood vessel comprising preparing a blood vessel for implantation in a patient and exposing the blood vessel to a physiological solution that comprises an exogenous substrate for an SSAO enzyme. Another embodiment is a composition having a concentration of an exogenous substrate for an SSAO enzyme, wherein the concentration of the exogenous substrate is at least great enough to relax a blood vessel exposed to the solution. Another embodiment is a medicament having an exogenous substrate for an SSAO enzyme. Another embodiment is a treatment for high blood pressure using an exogenous substrate for an SSAO enzyme.

### GOVERNMENT SUPPORT

The U.S. government has certain rights in this invention. Portions of this work were directly or indirectly supported by NIEHS Academic Research Enhancement Award Grant #1 R15 ES-011141-01, University of Wisconsin-Eau Claire Office of Research and Sponsored Programs, and the Ronald E. McNair Postbaccalaureate Scholar Program, NIH Grant #HL-65416, and NIA Grant #R03 AGI-8094.

### TECHNICAL FIELD

Aspects of the invention relate to amine-containing compounds that are vasorelaxants, and methods of making and using the same.

### BACKGROUND

Enzymes are biological molecules found in the body that catalyze chemical reactions so that a first chemical, termed a substrate, is chemically reacted to form other chemicals termed products. The substrates for a particular enzyme have common chemical features that allow the substrate to be processed by the enzyme.

The semicarbazide-sensitive amine oxidase (SSAO) enzyme is present in many tissues in the body, including the aorta, and other blood vessels. Many substrates for the SSAO enzyme are small molecules that are recognizable by their low molecular weight and the presence of a primary amine, which is a chemical group having the structure —NH₂.

The products of the SSAO enzyme have historically been thought to cause injuries to blood vessels and to be involved in vascular diseases such as atherosclerosis and diabetes (3, 5, 6, 19, 22, 27, 43, 49, 50, 52, 53). Indeed, it has even been suggested that blocking SSAO enzymes would be beneficial for human health (17,19,51,53).

### SUMMARY OF THE INVENTION

The inventor has discovered, contrary to suggestions in the literature, that SSAO substrates can be used without toxic effect as vasorelaxants have therapeutic uses. These SSAO substrates are converted by SSAO into products that, in turn, cause vasorelaxation. Vasorelaxation is helpful for treating various diseases and conditions, including vasospasm and high blood pressure (hypertension). Examples of SSAO substrates include methylamine, benzylamine, and compounds having the formula R—NH₂, where R is a hydrocarbon group (allyl or aryl) having about 12 or fewer carbons.

Further, vasorelaxation mediated by SSAO enzymes may be used to treat vasospasm, including those observed in surgical treatments wherein a donor blood vessel is to be implanted into a patient. The donor blood vessels can undergo a spasmodic contraction during the bypass procedures that can cause dangerous complications. But SSAO enzyme-mediated vasorelaxation can be used to relax the blood vessels prophylactically so as to avoid vasospasms. For example, the donor blood vessels can be bathed in solutions that have SSAO substrates so that the SSAO enzymes can cause vasorelaxation.

An embodiment of the invention relates to a method of processing a blood vessel that involves preparing a blood vessel for implantation in a patient and exposing the blood vessel to a physiological solution that comprises an exogenous substrate for an SSAO enzyme. Another embodiment relates to a composition having an in vitro blood vessel, a physiologically acceptable solution that includes a physiologically acceptable concentration of an exogenous buffer that provides a physiological pH, and a concentration of an exogenous substrate for an SSAO enzyme, wherein the concentration of the exogenous substrate relates to at least great enough to relax a blood vessel exposed to the solution. Another embodiment relates to a medicament comprising a purified exogenous substrate for an SSAO enzyme and a pharmaceutical carrier. Examples of SSAO substrates include methylamine, benzylamine, and equivalents thereof. Another embodiment is a medicament comprising a purified exogenous substrate for an SSAO enzyme and a pharmaceutical carrier. Another embodiment is a kit for treating a patient, the kit comprising a medicament containing an SSAO substrate and instructions for use of the medicament.

### DESCRIPTION OF THE FIGURES

FIG. 1 is a plot of data as discussed in the Examples showing that methylamine (MA) produced vessel specific responses in human blood vessels. A) Quantification of maximum MA-induced responses showed MA predominately relaxed LIMA (LIMA−; n=35), but produced a notable biphasic response, contraction first (+) followed by relaxation (−), in both RA (n=9) and RSV (n=21) although the relaxation in RA was significantly greater than that in RSV. B) Typical methylamine (MA, 1-1,000 μmol/L) cumulative concentration response curves (CRC) in norepinephrine-precontracted (NE, 1 μmol/L) isolated human blood vessels (left internal mammary artery, LIMA; radial artery, RA; right saphenous vein, RSV) from one patient. C) MA responses were plotted as percentage of the maximal response to determine the sensitivity of vessels to MA. The apparent effective concentration producing 50% response (apparent EC₅₀) was determined from CRC for both the MA-induced contractions (+) and relaxations (−) observed in NE-precontracted vessels. Values=means±SE. n=number of vessels. * or \=significant difference from other vessels of same category (i.e., + or −).

FIG. 2 is a plot of data as discussed in the Examples showing representative tracings of methylamine (MA, 1 mmol/L, 10 min) effects on norepinephrine-induced contractions (NE, 1 μmol/L) in left internal mammary artery (LIMA). Methylamine exposure occurred as a pre- or post-treatment to NE contraction. Occasionally, methylamine pretreatment slightly reduced baseline tension.

FIG. 3 is a plot of data as discussed in the Examples, wherein A) Representative tracings of responses of control and semicarbazide-pretreated (SEMI, 1 mmol/L, >15 min) isolated left internal mammary artery (LIMA) rings from one patient to norepinephrine (NE, 1 μmol/L), methylamine (MA, 1 mmol/L, 10 min), and acetylcholine (ACh, 1 μmol/L). B) SEMI significantly blocked methylamine-induced relaxation but had no effect on NE contraction and ACh relaxation in LIMA rings (n=8-9). Values=means±SE and are presented as a percentage of the control NE-induced contractions. n=number of vessels. *=significant difference with methylamine control.

FIG. 4 is a plot of data as discussed in the Examples showing the relationships between methylamine-induced relaxation (MA, 1 mmol/L, 10 min), acetylcholine-induced relaxation (ACh, 1 μmol/L), and age in isolated norepinephrine-precontracted (NE, 1 μmol/L) left internal mammary artery (LIMA; n=20) were investigated. A) The percentage methylamine-induced relaxation was not correlated with the percentage ACh-induced relaxation (R²=0.0001) or patient age, although ACh-relaxation and patient age were significantly correlated (R²=0.23; P=0.014). B) LIMA were pretreated with the nitric oxide synthase inhibitor, L-nitroarginine methylester (LNAME, 200 μmol/L, 20 min), indomethacin (INDO, 100 μM, 20 min), or LNAME+INDO. LNAME pretreatment significantly reduced ACh-induced relaxation in a subset of LIMA (n=9) with strong ACh-induced relaxations (ACh Control; ≧15% relaxation; ACh+) but had no effect on methylamine-induced relaxation (MA+LNAME). Values=means±SE. n=number of vessels. *=significantly different from all other treatments.

FIG. 5 is a plot of data as discussed in the Examples A) Semicarbazide-sensitive amine oxidase (SSAO) activity was similar between the homogenized human blood vessels (left internal mammary artery, LIMA, n=26; radial artery, RA, n=12; right saphenous vein, RSV, n=5). B) SSAO activity in human blood vessels was not correlated with the age of the patient (R²=0.0002). C) Semicarbazide (SEMI) inhibition of SSAO activity in homogenized human LIMA (n=26), RA (n=12), and RSV (n=5) was concentration-dependent, yet no differences between vessels in the SEMI concentrations producing 50% inhibition (IC₅₀) were detected. Blood vessel homogenates were preincubated with SEMI (1, 10, 100, and 1,000 μmol/L; 20 min) and assayed at 37° C. Values=means±SE and are presented as a percentage of control vessel SSAO activity (i.e., without SEMI). n=number of vessels.

FIG. 6 is a schematic diagram depicting an embodiment of an isolated blood vessel in an in vitro solution of an SSAO substrate.

### DETAILED DESCRIPTION OF THE INVENTION

The invention provides compositions and methods for relaxing blood vessels, including in vitro during medical procedures and in vivo as a medication, e.g., for blood pressure regulation. Semicarbazide-sensitive amine oxidase (SSAO) substrates may be used to relax a blood vessel. The SSAO enzymes naturally present in the blood vessel catalyze the reaction of the SSAO substrate to form metabolites that relax the blood vessel, or maintain it in a relaxed state. For example, a blood vessel taken from a patient for reimplantation into the patient as a heart bypass blood vessel may be exposed in vitro to a solution of an SSAO substrate. Consequently, the blood vessel stays relaxed and does not spasm so that the vessel may successfully be used for the bypass. And, for example, a medicament that comprises an SSAO substrate may be administered to a patient. As a result, a patient's blood pressure is reduced. Another embodiment is an SSAO substrate formed into a composition for exposure to a blood vessel to thereby mediate relaxation; for example, such a composition could be made available through a scientific reagent supply company catalog. Another embodiment is a composition that contains exogenous SSAO substrate that may be administered in vitro or to a patient.

Exposing a blood vessel to an exogenous SSAO enzyme substrate allows the substrate to interact with SSAO endogenous to the blood vessel and be converted into SSAO products, which include metabolites that cause relaxation of the blood vessel. When a blood vessel is placed into a solution of SSAO substrates, the substrates continuously diffuse into the blood vessel and react with the endogenous SSAO enzyme so that a continuous output of relaxing metabolites is maintained. SSAO substrates spread through the three dimensional structure of the blood vessel react with the enzyme to produce metabolites throughout the blood vessel. Moreover, SSAO enzymes are present in the smooth-muscle containing portions of some major blood vessels so that the SSAO metabolites are delivered directly to smooth muscle cells for desired relaxation effects. In contrast, a metabolite added directly to a solution holding the blood vessel may not penetrate deep into the three dimensional structure of the vessel if the metabolite has a short half-life in the solution relative to the time needed to diffuse into the vessel, e.g., H₂O₂. Or, a superficially introduced SSAO substrate may be consumed at outer tissue layers before it could penetrate into smooth-muscle cell containing layers.

It is conventionally thought that methylamine and SSAO enzyme activity contributes to cardiovascular disease, e.g., in human diabetics. The inventor had previously established that one of the substrates for SSAO enzyme is methylamine (MA), which has the structure CH₃NH₂ (54), and had also suggested that methylamine might be toxic (55). To further investigate the role of SSAO substrates, a number of additional studies were undertaken, which are described herein. Surprisingly, it was discovered that SSAO enzymatic activity and its products beneficially cause relaxation of blood vessels, so that SSAO substrates or products can be used as therapeutics to mediate these effects.

These results were generated using several experimental studies. Some of the studies measured the acute vasoactive effects of methylamine in isolated human blood vessels used for coronary artery bypass grafts, (CABG), including these blood vessels: the left internal mammary artery (LIMA); the radial artery (RA); and the right saphenous vein (RSV). Another set of studies demonstrated that methylamine's vasoactive effects were dependent on SSAO activity; using the SSAO inhibitor semicarbazide. Further studies determined the effects of methylamine metabolites formaldehyde and hydrogen peroxide in LIMA and RSV. Another set of studies tested whether the methylamine response was nitric oxide-, prostaglandin-, or hyperpolarization-dependent. Other studies herein quantified the LIMA and RSV cGMP levels following methylamine exposure. Additional experiments quantified SSAO activity in LIMA, RA, and RSV. The SSAO enzyme-mediated relaxation was robust, repeatable, reversible, and dependent on SSAO enzyme activity. Relaxations were not correlated with patient age or endothelium function. These data show that SSAO-induced vascular effects are dependent upon vascular SSAO-generated activity and, therefore, dependent on one or more of SSAO products.

A conventionally accepted hypothesis states that chronic methylamine (MA) exposure induces vascular injury and promotes vascular disease, including atherosclerosis, in humans via SSAO mediated metabolism of methylamine to injurious metabolites: formaldehyde, hydrogen peroxide (H₂O₂), and ammonia (NH₃) (19,50,52,53). Scientists who have performed clinical and experimental studies have supported such a relationship. Altered plasma methylamine levels, methylamine excretion, and elevated plasma SSAO activity are present in human diseases associated with chronic vascular pathology (e.g., diabetes mellitus, uremia; 3,5,6,27,49; see reviews 19,53). In the case of Type I diabetes, SSAO plasma levels increase at the onset of disease (6) and plasma SSAO activity positively correlates with the amount of glycosylated hemoglobin, an indicator of the severity of complications in human diabetics (5,43). Similarly, plasma SSAO activity is elevated within 2 weeks following streptozotocin-induced diabetes in rats (22). Thus, conventional understandings link methylamine levels and SSAO activity to the development of vascular pathology in diabetic humans, and recently the suggestion has been made that therapeutic inhibition of SSAO could slow the progression of vascular disease (17,19,51,53).

Methylamine is a common primary amine derived from a multitude of sources, and is preferentially metabolized by SSAO in comparison with other amine oxidases (16,33,47). Methylamine is both an exogenous (present in cigarette smoke and wine and foods) and an endogenous amine, and it is a metabolic end product of diverse compounds including epinephrine, carbamate insecticides, creatine, nicotine, and sarcosine (35,38,48). Methylamine metabolism in rats and humans appears to be due largely to SSAO activity (16,34). For example, excretion is elevated in rats following the administration of SSAO inhibitors (34), and in humans following consumption of creatinine, certain fish and seafood, and some fruits and vegetables (38). Moreover, methylamine is metabolized by vascular homogenates, including rat aorta and human umbilical artery, to formaldehyde (9,39). Finally, SSAO inhibitors prevent toxicity in cultured endothelial cells (47). Thus, present evidence supports the concept that methylamine, whether exogenous or endogenous, is converted to metabolites, formaldehyde and H₂O₂, by endogenous SSAO activity.

The plasma and tissue forms of the SSAO (e.g., EC 1.4.3.6) enzymes are distinct from the monoamine oxidases (MAO), diamine oxidases, and polyamine oxidases (33). The copper-containing SSAOs share common features including insensitivity to MAO inhibitors (e.g., clorgyline, deprenyl), preference for aliphatic amines and the aromatic benzylamine, and inhibition by carbonyl-containing compounds, such as semicarbazide, for which the most current name is derived (33,47). SSAO activity is present in all mammalian cardiovascular tissues tested, including plasma/serum, aorta, and heart with the most concentrated SSAO activity present in the mammalian aorta, including human and rat (11,13,14,32,33,37,39,44). This high level of SSAO activity in the cardiovascular tissues implies functionality, although a specific function for the SSAO enzyme has yet to be conventionally established (8,33,53).

The vascular effects of methylamine were studied in isolated human blood vessels, as reported herein. It has been discovered that SSAO enzymatic activity and SSAO-produced metabolites beneficially cause relaxation of blood vessels, so that SSAO substrates or products can be used to mediate these effects.

Several of the findings herein are contrary to the conventionally understood role of methylamine as a potential vascular toxicant in humans. One finding herein is that a prominent effect of an SSAO substrate such as methylamine in isolated human blood vessels is a generally robust yet benign relaxation. This relaxation, highly expressed in LIMA, is dependent on vascular SSAO activity, and significantly, is reversible and repeatable. It is quite distinct from the SSAO-dependent, yet quite injurious, actions of allylamine, a well-known cardiovascular toxicant, or the α, β-unsaturated aldehyde, acrolein, in isolated rat thoracic aorta, rat coronary arteries, and human blood vessels where both agents produce vasospasm in rat coronary arteries. The findings reported herein surprisingly show that methylamine may be a source of vasoactive signaling molecules via vascular SSAO activity.

In the present study, methylamine at 1-1,000 μM had no observable adverse effects in isolated human blood vessels. However, it is clear that very high methylamine exposure can be lethal in humans. As a result of an accidental spill of purified liquid methylamine, 35 Chinese persons of 7-71 years of age and nearly equal male/female distribution were hospitalized 7-8 hours post-exposure with 6 resulting fatalities (46). Overt toxicity in these patients included significant cardiovascular symptoms including tachycardia and unmeasurable (low) blood pressure and pulse. These symptoms are consistent with severe systemic hypotension, accompanied by reflex tachycardia, declining cerebral perfusion, and ultimate coma (10 of 35 people suffered light to deep coma). Based on the findings herein, and in light of the widespread presence of SSAO enzyme in human conductance and resistance blood vessels, it can be understood that the systemic, high-level dose of methylamine caused prolonged and robust blood vessel relaxation that caused severe hypotension, as indicated by the observations of severe low blood pressure and tachycardia, see also 11, 14, 23, 32, 39.

With the exception of the unusual circumstances of this accident in China, a toxic-level dose of methylamine in humans is unlikely. Although methylamine is present in a variety of common exogenous sources, including wine, cigarette smoke, and as a metabolite of nicotine (12), it is unlikely that one could reach acute toxic doses by these paths (35, 38, 48), since estimated normal human plasma methylamine concentration is less than about 1-5 μmol/L, and in uremic human plasma methylamine concentration is about 10-20 μmol/L (3, 45). Some red wines possess up to 5 mg/l methylamine (35) which could elevate plasma methylamine levels by about 30 μmol/Lin a 70-kg male assuming consumption of 1 liter of wine and 100% absorption.

It has been suggested that treatment of human diabetics with an SSAO inhibitor, such as aminoguanidine, may provide vascular protection by inhibiting SSAO activity, diminishing amine metabolism, subsequent aldehyde and adduct formation, and reducing advanced glycation end products (AGEs) (17, 20, 37, 48). However, it is unclear how a SSAO inhibitor would affect the variety of cardiovascular and non-cardiovascular pools of SSAO activity including adipose and G.I. tract smooth muscle where the function of SSAO also remains undetermined (18). For example, the recent identification of the SSAO protein homolog, vascular adhesion protein-1 (VAP-1) expressed in endothelial and vascular smooth muscle cells is involved in lymphocyte binding (26, 41). While previous studies detect little to no SSAO activity in endothelial cell cultures (50), purified human and bovine brain microvessels possess concentrated SSAO activity with relatively high affinity for methylamine (11). Thus, without being bound to a particular hypothesis, endothelial cell VAP-1 may contribute to overall vascular SSAO enzymatic activity and/or to non-enzymatic functions associated with amine binding. Moreover, there are no specific inhibitors for each tissue-specific pool of SSAO activity (17, 37). Furthermore, the effects of SSAO inhibition or overexpression in developing rats are detrimental to vascular tissue (19, 29) and remain unknown in the adult, although treatment of Parkinson's patients with carbidopa or hydralazine SSAO inhibitors, may indicate limited effects in adults (see 19, 53).

SSAO substrates must have specific chemical and structural motifs to function as an SSAO enzyme substrate. SSAO, like other enzymes, interacts only with chemicals having appropriate chemical or structural motifs. A chemical can readily be tested to determine if it is an SSAO substrate using techniques known to those of ordinary skill, e.g., as demonstrated in the Examples herein, or as in Yu (1990) (47).

Methylamine, which has the formula CH₂NH₂, is an SSAO substrate. A variety of compounds are known to be substrates for SSAO, for example as set forth in Yu (1990) (47). Many of the motifs for SSAO can be predicted based on analogy to the specific chemicals that are known to be SSAO substrates. Medicinal chemistry techniques and tools known to persons of ordinary skill can be used to suggest variations of known substrates and to predict new substrates that are reasonably expected to have structures that function as SSAO substrates. Moreover, embodiments include compounds that are processed by SSAO enzymes to produce at least one of the metabolites that methylamine produces when methylamine is metabolized by SSAO, e.g., H₂O₂, formaldehyde.

Certain substrates can therefore be described as having a formula of R₁—X₁—NH₂, wherein R₁ is chosen from a group consisting of NH₂, H, OH, and COOH, and wherein X₁ is an alkyl having between one and twelve carbons, X₁ is a C₆ aromatic ring, or X₁ comprises a single C₆ aromatic ring and further comprises between one and eleven alkyl carbons. For example, X₁ may be CH₂. Or, for example, X₁ may be CH₂, and R₁ may be H, whereby the substrate has the formula CH₃NH₂. Or, for example, X₁ may include a C₆ aromatic ring. Or, for example, X₁ may include a C₆ aromatic ring while R₁ is H.

Certain SSAO substrates may be described by the formula 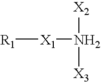
wherein R₁ is NH₂, H, OH, or COOH; wherein X₂ is NH₂, H, OH, COOH, or an alkyl having between one and three carbons; wherein X₃ is NH₂, H, OH, COOH, or an alkyl having between one and three carbons; and wherein X₁ is an alkyl having between one and twelve carbons, or X₁ is a C₆ aromatic ring, or X₁ includes a single C₆ aromatic ring and further includes between one and eleven alkyl carbons.

Moreover, the metabolite produced by an SSAO enzyme and a particular SSAO substrate can be readily determined using techniques known to those of ordinary skill in these arts. Another embodiment relates to a solution, medicament, or composition of an SSAO substrate that produces H₂O₂ in the presence of an SSAO enzyme, e.g., an SSAO enzyme found in a blood vessel tissue.

Another embodiment relates to a solution of an exogenous SSAO substrate that contacts a blood vessel taken from a patient. The solution has a concentration of exogenous SSAO substrate that is sufficient to cause relaxation or to prophylactically maintain relaxation of the vessel. Exogenous refers to a material that is not naturally present. For example, a blood vessel naturally contains some SSAO substrate; this naturally present substrate is not exogenous. Another embodiment relates to a solution an exogenous SSAO substrate present at a concentration in the range of about 0.01 to about 1000 millimolar, or any concentration or range therebetween, for example, from 0.01 to 100 millimolar, and from 0.1 to 10 millimolar. A person of ordinary skill in the art will recognize that additional ranges of concentrations are contemplated and are within the present disclosure. Many examples of SSAO substrates are provided herein.

Certain embodiments relate to the administration of an SSAO substrate, e.g., methylamine, to a patient, e.g., a human, for a therapeutic purpose, e.g., to lower blood pressure. Examples of doses are, e.g., 10-10,000 mg/kg, at least 10 mg/kg, and less than 10,000 mg/kg. A person of ordinary skill in these arts will realize that all ranges and values within the range specifically set forth are contemplated and included herein, e.g., 10-100 mg/kg, at least 20 mg/kg, and less than 1,000 mg/kg. Moreover, values outside of the explicitly stated range are also contemplated.

Administration of such substrate(s) for lowering blood pressure may be adapted to the particular medical condition that is being addressed. For example, chronic high blood pressure may require long-term dosage regimens. Transient high blood pressure, on the other hand, could be treated with a short-term dosage regimen. Moreover, such substrate(s) may generally be used in combination with conventional medications for treating blood pressure.

An SSAO substrate may be combined with other SSAO substrates. Since some substrates have faster reaction times than others, or have different half-lives in solution or in a patient, there may be advantages in making certain combinations. For example, methylamine can be combined with other SSAO substrates in a solution or medicament. Certain embodiments include at least one SSAO substrate combined with a metabolic product of the SSAO process. An advantage of such a combination would be to achieve an immediate effect through the metabolite and a longer term effect through the SSAO substrate. An example of an SSAO metabolite is H₂O₂.

An SSAO substrate may also be combined with conventionally used relaxants. The SSAO substrate could produce longer term relaxation, affect smooth muscle cells more directly, or complement other metabolic pathways. An example of a conventional relaxant is papaverine, which is used to treat vasospasms of excides vessels in vitro. Other conventional relaxants are blood pressure medications, e.g., diuretics, beta-blockers, ace inhibitors, angiotensin antagonists, calcium channel blockers, alpha-blockers, alpha-beta-blockers, nervous system inhibitors, and vasodilators. Specific high blood pressure drugs are known, e.g., alphamethyldopa, clonidine, doxazosin, guanabenz, guanadrel, guanethedine, guanfacine, hydralazine, mecamylamine, minoxidil, phenoxybenzaline, prazosin, reserpine, and terazosin. Any SSAO substrate, conventional relaxant, and metabolite described herein may be combined in any combination.

The exposure time that results in a desired level of relaxation of a blood vessel in contact with an SSAO substrate may vary according to the concentration of the exogenous substrate and its chemical composition. Some SSAO substrates are catalyzed more quickly than others. The time to achieve desired effects can be determined by following testing protocols described in the examples or by other means known to those skilled in these arts after reading this disclosure. Many working embodiments, however, can be expected to have an exposure time for initial visual observation of vascular relaxation of between about one minute and about 15 minutes. Embodiments include exposing a blood vessel to an exogenous SSAO substrate for at least about one minute, including at least about 2, about 5, about 10, about 15, about 30 or about 60 minutes. Since a blood vessel typically comprises living cells that are optimally exposed to in vitro conditions for a limited time, a maximum time can reasonably be planned for many embodiments. A blood vessel in vitro refers to a blood vessel that has been separated from a patient and is not been reimplanted. Thus, some embodiments include exposing a blood vessel to an exogenous SSAO substrate for a time that ranges from about 1 minute to about 300 minutes and all ranges therebetween, e.g., from about 5 to about 60 minutes, and from about 15 to about 90 minutes. A person of ordinary skill in the art will recognize that additional ranges of exposure times are contemplated and are within the present disclosure.

Another embodiment relates to a composition that comprises an exogenous SSAO enzyme. The composition may be used in vitro, e.g., with blood vessels, or administered to a patient. The composition may include other embodiments as described herein, or combinations thereof, e.g., SSAO substrates, conventional relaxants, and pharmaceutical carriers. The exogenous SSAO enzyme for humans is reported in Moldes et al., J. Biol. Chem. 274:9515-9523 (1999). Exogenous SSAO enzymes include all human and mammal SSAO enzymes and alternative forms of the enzyme that have similar functionality, including portions, active sequences, natural mutants, and human-engineered variants. Examples of dosages for a human are from 1 to 100 mg/kg; a person of ordinary skill in these arts will recognize that all possible values and ranges therebetween are contemplated, as well as other doses outside the explicitly stated range. See also Kumar et al., J Toxicol Sci. 14:105-14. (1989).

Another embodiment relates to a physiologically acceptable solution of an SSAO substrate. Physiologically acceptable solutions have an osmolarity and pH that are compatible with blood vessels, and preferably have a pH of between about 7.0 and about 7.8 and an osmolarity of between about 280 and about 350 milliOsmoles. Alternatively, other pH and osmolarities may be suitable, and may vary according to the particular solution or application, and the length of exposure to the solution. Some embodiments have a pH in the range of about 6 to about 8, e.g., from 6.3 to 7.8, and from 6.7 to 7.6. Such solutions may be buffered with a biocompatible buffer having a buffering capacity within the about 7 to about 7.8 range, e.g., phosphates, and bicarbonates. A variety of physiologically acceptable solutions are commercially available.

Certain embodiments are related to medicaments. Medicaments are compositions that are pharmaceutically acceptable and contain active ingredients and controlled amounts and types of other ingredients, with the exception of trace impurities. Thus a medicament has a known composition. A medicament is therefore distinguishable from naturally-occurring compounds and substances such as wine that are not fully characterized. The phrase pharmaceutically acceptable refers to those compounds, materials, compositions, and/or dosage forms which are, within the scope of sound medical judgment, suitable for use for consumption by human beings, or being in contact with the tissues of human beings and animals without excessive toxicity, irritation, allergic response, or other problem or complication, commensurate with a reasonable benefit/risk ratio.

Embodiments of compounds set forth herein may also be prepared as pharmaceutically acceptable salts. The phrase pharmaceutically acceptable salts refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids.

Lists of suitable carriers, excipients, and salts are found in, for example: Remington: The Science and Practice of Pharmacy, 18th ed., by Alfonso R. Gennaro, Mack Publishing Company, Easton, Pa., 1990; Pharmaceutical Dosage Forms and Drug Delivery Systems, by Ansel, Popovich and Allen Jr., Lippincott Williams & Wilkins Publishers; 7th edition (July 2004); and Handbook of Pharmaceutical Excipients by Arthur H. Kibbe (Editor), Ainley Wade, Paul J. Weller, 3rd edition (Jan. 15, 2000); the disclosures of which are hereby incorporated by reference.

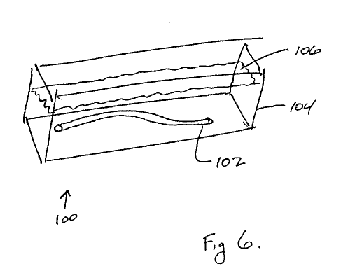

An embodiment relates to a biologically acceptable composition comprising an SSAO substrate. The composition may be, for example, mixed in a biologically acceptable carrier suitable for administration to a blood vessel. For example, a known amount of the SSAO substrate may be contained in the biologically acceptable composition. A medicament is an example of a biologically acceptable composition. Moreover, a pharmaceutically acceptable composition is also a biologically acceptable composition. Embodiments include adding a biologically acceptable composition of an SSAO substrate to an in vitro container. For example, FIG. 6 depicts a system 100 for relaxing a blood vessel 102 that includes container 104 having a physiological solution of SSAO substrates 106.

An embodiment includes preparing a pharmaceutically acceptable medicament that comprises an SSAO substrate. Another embodiment relates to a medicament with a therapeutically-effective amount of an SSAO substrate. Another embodiment relates to an SSAO substrate formulated together with one or more pharmaceutically acceptable carriers (additives) and/or diluents. As described, below, Another embodiment relates to a pharmaceutical composition formulated for administration in solid or liquid form, including those structured for: (1) oral administration, e.g., drenches (aqueous or non-aqueous solutions or suspensions), tablets, boluses, powders, granules, pastes; (2) parenteral administration, e.g., by subcutaneous, intramuscular or intravenous injection, for example, in a sterile solution or suspension; (3) topical application, e.g., as a cream, ointment or spray applied to the skin; or (4) intravaginally or intarectally, e.g., as a pessary, cream or foam. Also, for example, an aerosol, mist, atomizing solution, surgical glue, medical tape, or patch may be used as a medicament with other embodiments disclosed herein.

The phrase pharmaceutically acceptable refers to compositions which, within the scope of sound medical judgment, are suitable for use in contact with the tissues of human beings and animals without excessive toxicity, irritation, allergic response, or other problem or complication, commensurate with a reasonable benefit/risk ratio. A pharmaceutically-acceptable carrier refers to a pharmaceutically-acceptable material, composition or vehicle, such as a liquid or solid filler, diluent, excipient, solvent or encapsulating material, involved in carrying or transporting the subject compound from one organ, or portion of the body, to another organ, or portion of the body. Examples of materials which can serve as pharmaceutically-acceptable carriers include: sugars, starches, cellulose, malt; gelatin; talc; excipients, such as cocoa butter and suppository waxes; oils, glycols, such as propylene glycol; polyols, polyethylene glycol, esters, such as ethyl oleate and ethyl laurate; agar; buffering agents, alginic acid; pyrogen-free water, isotonic saline; Ringer's solution; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Methods of preparing these formulations or compositions include the step of bringing into association a composition as described herein with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product. Formulations of the invention suitable for oral administration may be in the form of capsules, cachets, pills, tablets, powders, granules, or as a solution or a suspension in an aqueous or non-aqueous liquid, or as an oil-in-water or water-in-oil liquid emulsion, or as an elixir or syrup, or as pastilles, each containing a predetermined amount of composition as described herein as an active ingredient.

A composition as described herein may also be administered as a bolus, electuary or paste. In solid dosage forms of the invention for oral administration (capsules, tablets, pills, dragees, powders, granules and the like), the active ingredient may be mixed, e.g., with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, polyethylene glycols and fatty acid esters of sorbitan, and mixtures thereof.

A composition as described herein may be administered to humans and other animals for therapy by any suitable route of administration, including intravenously, orally, nasally, as by, for example, a spray, rectally, intravaginally, parenterally, intracistemally and topically, as by powders, ointments or drops, including buccally and sublingually. For example, an SSAO substrate, e.g., methylamine, may be inhaled as a vapor or administered by inhalation using a metered device. Regardless of the route of administration selected, a composition as described herein, which may be used in a suitable hydrated form, and/or the pharmaceutical compositions of the present invention, are formulated into pharmaceutically-acceptable dosage forms by conventional methods known to those of skill in the art. If desired, the effective daily dose of the active compound may be administered as two, three, four, five, six or more sub-doses administered separately at appropriate intervals throughout the day, optionally, in unit dosage forms.

While it is possible for a SSAO substrate as described herein to be administered alone, it may be administered as a pharmaceutical formulation (composition). A composition as described herein may be formulated for administration in any convenient way for use in human or veterinary medicine, by analogy with other pharmaceuticals. A patient receiving a treatment is any animal in need or under investigation, including primates, in particular humans, and other mammals such as equines, cattle, swine and sheep; and poultry and pets in general.

Compositions may be made with a suitable weight of SSAO substrate. For example, a composition including an SSAO substrate may be made for use as a biologically acceptable composition, a medicament, or a reagent for research. For example, for such a use, a composition may be made having at least one mg, or between 0.1 and 10,000 mg of an SSAO substrate, including all values and ranges therebetween, e.g., 1 to 10,000 mg, 10 to 100 mg, and 50 to 500 mg.

Kits may be made having at least one SSAO substrate, e.g., in a biologically acceptable form, and instructions for use. Instructions may include uses as described herein, e.g., for blood vessel relaxation, for making a solution for blood vessel relaxation for treating in vitro blood vessels, and as a blood pressure treatment. Instructions are communications that explain a use of a content of the kits. Instruction formats include, e.g., writing, audio, electronic, web-interactive, email, label, brochure, slide, or handout. For example, a kit having an SSAO substrate may comprise a container of the substrate and a presentation made by an entity directly or indirectly selling the SSAO substrate to a user of the container of SSAO substrate by slide, speech, brochure, commercial, or website.

A method of using an SSAO substrate includes at least one of the steps of purifying a source of an SSAO substrate, packaging the SSAO substrate as a biologically acceptable composition, ensuring the quality of the composition, testing the composition for biological acceptability, providing a written statement of the contents of a container of the composition, stocking the composition, ordering the composition, stocking the composition locally, distributing the composition for use, and end-use of the composition.

### EXAMPLES

### Materials and Methods

Human Subjects and Blood Vessels Consenting adult humans (age yrs, mean±SE, all, 62.8±1.8; males, 59.9±2.4, ≈77% of total; females, 71.9±1.9) undergoing coronary artery bypa graft (CABG) surgery at Luther Hospital/Midelfort Clinic (Eau Claire, Wis.) between 2000 and 2003 were the source of the blood vessels (IRB#T-4028). Unused sections of left internal mammary artery (LIMA), left and right radial arteries (RA), and right saphenous vein (RSV), were placed in lactated Ringers and refrigerated (4° C.) at the hospital. Vessels were retrieved between 4-16 hrs after surgery, cleaned of blood, staples, thread, and extraneous tissue, and placed in fresh physiological saline solution with glucose (PSS; pH 7.4; 4° C.). All vessel experiments were begun within 24 hrs of surgical removal.

Vascular Ring Physiology Human blood vessel segments used were free of overt trauma and relatively free of luminal thrombi and adventitial hematomas. The majority of vessels of each vessel type were uniform in size but there was variation and the exact location from whence each vessel segment was removed was unknown (e.g., ankle vs. knee region of RSV). The segment ends were trimmed and ˜2-3 mm segments (‘rings’) were cut. Rings were hung on stainless steel hooks in PSS bubbled with 20%:5% O₂:CO₂ at 37° C. One hook was connected to an isometric strain gauge transducer (Kent Scientific; Litchfield, Conn.), while the other was attached to a fixed support rod. Transducer signals were fed into a PowerLab A/D converter and recorded on a PC using Chart software (v. 3.4.9; iWorx, Dover, N.H.). Aortas (rat TA) were taken from CO₂ euthanized 12-14 week old male Sprague-Dawley rats and approximately 1-yr old male Wistar rats, placed in cold PSS, and treated as previously reported (14).

All rings were subjected to the same four initials steps in sequence. Rings were equilibrated to a specified tension for 30 min in the bath (1 g for RSV and rat TA or 3 g for LIMA and RA; 15). Rings were stimulated with 100 mmol/Lpotassium-PSS (HI K⁺) to test for viability. Rings were washed 3 times with PSS over 30 min, and re-equilibrated to resting tension (3×PSS). Rings were contracted with norepinephrine (NE, 1 or 10 μmol/L, Control NE) and then stimulated with acetylcholine (ACh, 1 μmol/L) to test for the presence of an endothelial-derived relaxing factor (EDRF; nitric oxide, NO.) response before experimentation (i.e., a contraction and relaxation cycle; C/R).

Following the standard protocol, each ring was assigned one of four experimental protocols: 1) Unstimulated rings were exposed to 1 mM methylamine (10 min) or cumulative methylamine, formaldehyde, or H₂O₂ concentration (1 μM, 10 μM, 0.1 μM, and 1 mM) followed by a C/R cycle; 2) NE-precontracted rings were exposed to 1 mM methylamine, formaldehyde, or H₂O₂ (10 min) or cumulative methylamine, (1 μM, 10 μM, 0.1 μM, and 1 mM) before or after ACh; 3) semicarbazide (SEMI, 1 mM, 10 min) pretreated rings were contracted with NE followed by exposure to 1 mM methylamine then ACh addition (in LIMA only due to availability); or 4) N^{ω}-nitro-L-arginine methyl ester (L-NAME, 200 μmol/L, 20 min), indomethacin (INDO, 100 μmol/L, 20 min), or L-NAME+INDO pretreated rings were stimulated with a C/R cycle followed by exposure to 1 mmol/LMA (in LIMA only).

After all treatments, and 3×PSS washouts, rings were precontracted with HI K⁺ followed by exposure to ACh (1 μmol/L), and then sodium nitroprusside (SNP; 100 μmol/L). ACh and SNP exposure assessed vessel responsiveness to endogenous and exogenous NO, respectively. To test for methylamine hyperpolarization, a subset of HI K⁺-precontracted rings were exposed to methylamine or H₂O₂ (1 mmol/L; 10 min) followed by ACh and SNP addition (in LIMA only). Experimental duration was typically 4-6 hrs.

Vessel contractions were normalized as a percentage of the control NE contraction. Vessel relaxations were calculated as the percentage reduction of the agonist-induced contractions (i.e., NE or HI K⁺). Cumulative concentration response curves were used to interpolate the apparent effective concentration producing 50% relaxation (NE-precontracted vessels) or contraction (uncontracted and NE-precontracted vessel % responses were pooled). Relaxation half-times (t_{1/2} in sec) were calculated for methylamine (1 mmol/L) and ACh (1 μmol/L) relaxations in NE-precontracted LIMA and for SNP (100 μmol/L) relaxations in HI K⁺ precontracted LIMA.

cGMP ELISA Assay; Vessels and Stimulation Protocol: To ascertain if cGMP was involved in the methylamine relaxation, we used longer segments of LIMA and RSV (0.5-1.0 cm; 100-150 mg total) exposed to methylamine or SNP. The percentage relaxation and cGMP level for each vessel were quantified. Briefly, vessel segments were contracted with HI K+ solution, followed by 3×PSS, and a C/R cycle (as above). Next, they were precontracted with 1 or 10 μmol/L NE and when stable exposed to methylamine (1 mmol/L) or SNP (100 μmol/L). After allowing the response to plateau (20-40 min), vessels were wrapped in aluminum foil, frozen in 1N2, and stored at −80° C. until analysis. Unstimulated LIMA and RSV (n=6 patient matched) vessels were used for baseline measurement of cGMP.

Vessel segments were minced on ice and cold homogenized in 0.1 mol/L HCl (25-30 strokes; 0.1 g vessel wet weight/1 ml HCl). A homogenate subsample (50 μl) was frozen for protein analysis and remaining samples were centrifuged (14,000×g; 20 min). Supernatants were processed for cGMP analysis according to the ELISA kit manufacturer's instructions (DirectCyclic GMP Kit; Assay Designs, Inc., Ann Arbor, Mich.) using the acetylated and overnight incubation protocols. Protein was determined with the Bio-Rad Protein Dye Concentrate reagent (Bio-Rad, Hercules, Calif.) using bovine serum albumin as standard (Sigma Chem. Co., St. Louis, Mo.).

Semicarbazide-Sensitive Amine Oxidase (SSAO) Assay Standard assay protocols for measuring SSAO activity radiometrically using ¹⁴C-benzylamine-hydrochloride as substrate were followed (BZA, 1 μmol/L; 59 μCi/mmol; Amersham Inc., Rockford, Ill.; 14,32). Blood vessel segments were homogenized in Sorensen's Na⁺/K⁺-phosphate buffered solution at a ratio of 1 g sample per 30 ml buffer using a hand-held glass homogenizer (PBS, 0.1 M, pH 7.8). The homogenate was centrifuged and 30 μl supernatant was used in each assay. SSAO final assay volume was 245 μl with ¹⁴C-BZA in PBS (1 μmol/L) and deprenyl in PBS (1 μmol/L). Following 30 min incubation at 37° C., the assay was stopped by addition of 2 mmol/Lcitric acid (150 μl). The assay volume was extracted (extraction efficiency assumed at >95% with no correction) with toluene:ethyl acetate (1:1, 1 ml), and an aliquot (100 μl) of the organic layer was counted (3 min) by liquid scintillation (Beta-fluor; National Diagnostics, Atlanta, Ga.). All samples were run in duplicate or triplicate.

SSAO activity was measured in homogenized human LIMA (mean patient age yrs, 66.1±2.0, n=27), RA (58.1±2.5, n=12), and RSV (66.4±0.6, n=5) with and without semicarbazide (SEMI, 1, 10, 100 or 1,000 μmol/L, 20 min preincubation) at 37° C. Protein was determined with the Bio-Rad Protein Dye Concentrate reagent (Bio-Rad, Hercules, Calif.) using bovine serum albumin in saline as standard (Sigma Chem. Co., St. Louis, Mo.). SSAO activity was calculated as the nmoles BZA substrate metabolized per 30 min per mg protein. SEMI inhibition was calculated as a percentage of the control SSAO activity (i.e., without SEMI=100%).

Chemicals and Solutions PSS was composed of the following in mmol/L: NaCl, 130; KCl, 4.7; MgSO₄.7H₂O, 1.17; KH₂PO₄, 1.18; NaHCO₃, 14.9; CaCl₂, 2.0; glucose, 5.0; pH 7.4. HI K⁺ PSS was composed of the following in mmol/L: NaCl, 34.7; KCl, 100; MgSO₄.7H₂O, 1.17; KH₂PO₄, 1.18; NaHCO₃, 14.9; CaCl₂, 2.0; glucose, 5.0.; pH 7.4. All other chemicals were purchased from Sigma Chemical Co. (St. Louis, Mo.) and dissolved in distilled water, except indomethacin which was dissolved in 0.1 mol/LNaHCO₃ in 0.1 N NaOH.

Statistics Values are reported as means±standard error of the mean (SE). Statistical comparisons between two groups were performed with Students paired or unpaired t-tests and between more than two groups using One-way ANOVA with post-test comparisons using the Student-Newman-Keuls test (SigmaStat, SPSS, Inc., Chicago, Ill.). Statistical significance was assumed at P≦0.05.

SSAO Substrate and Metabolite Effects in Isolated Human Blood Vessels

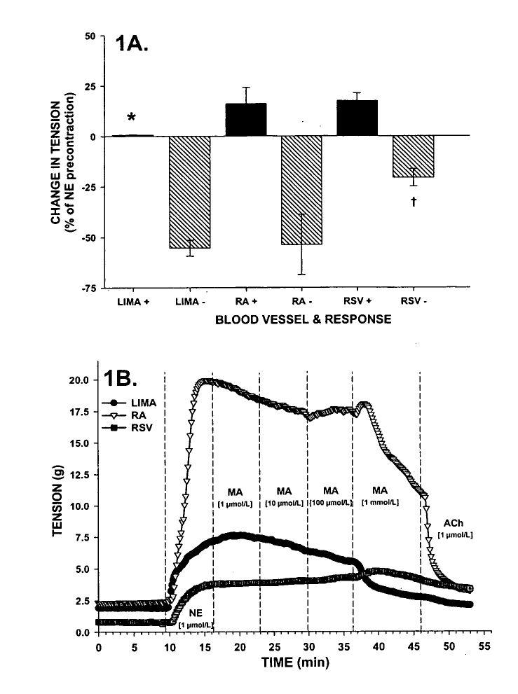

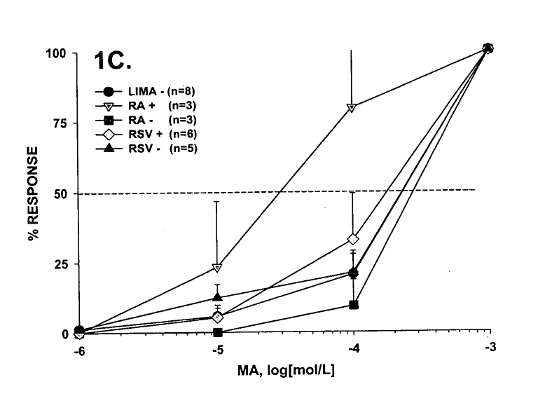

This Example includes data showing that SSAO substrates can mediate blood vessel relaxation. Methylamine (1 mM) exposure in NE-precontracted vessels produced a biphasic response, i.e., initial contraction (+) followed by prolonged relaxation (−), in RA and RSV and predominately a strong relaxation in LIMA (4 of 30 LIMA were weakly contracted; FIG. 1A). Methylamine-induced contractions in RA and RSV were significantly greater than in LIMA, while methylamine-induced relaxations were significantly greater in LIMA and RA than in RSV (FIG. 1A, Table 1).

Unlike human vessel responses to 1 mmol/L methylamine, NE-precontracted rat TA were minimally affected by methylamine and these responses appeared further blunted in TA of older rats. In contrast, vigorous biphasic responses to 1 mmol/L H₂O₂ were present in both young and old rat TA alike and were similar to the 1 mmol/L H₂O₂-induced responses in human LIMA (Table 1). Formaldehyde (1 mmol/L; 10 min) produced either minimal contraction or relatively strong relaxation while no biphasic responses were observed in any blood vessel tested (Table 1). Formaldehyde-induced relaxations were slow in onset, prolonged in duration, and overt toxicity was seen in only 2 of 18 rat TA and in none of the 6 human vessels exposed i.e., toxicity is the near complete loss of a tension response to HI K⁺ after 3×PSS washout following the formaldehyde exposure.

Vessel sensitivities to methylamine, formaldehyde, or H₂O₂ were comparable between toxicants and between vessels (FIG. 1C; Table 2). The RA were significantly more sensitive to methylamine-induced contraction than to methylamine-induced relaxation whereas LIMA were more sensitive to H₂O₂-induced relaxation when compared to RSV (Table 2). Similarly, RSV were more sensitive to methylamine-induced relaxation than to H₂O₂-induced relaxation (i.e., FIG. 1BC; Table 2). Of the 6 human vessels tested (3 LIMA and 3 RSV), no vessel responded to formaldehyde at less than 1 mmol/L (note the same apparent EC₅₀ values for all formaldehyde-induced responses; Table 2).

SSAO Substrate Mediated Relaxation in Isolated Human Blood Vessels: Toxicity and Repeatability

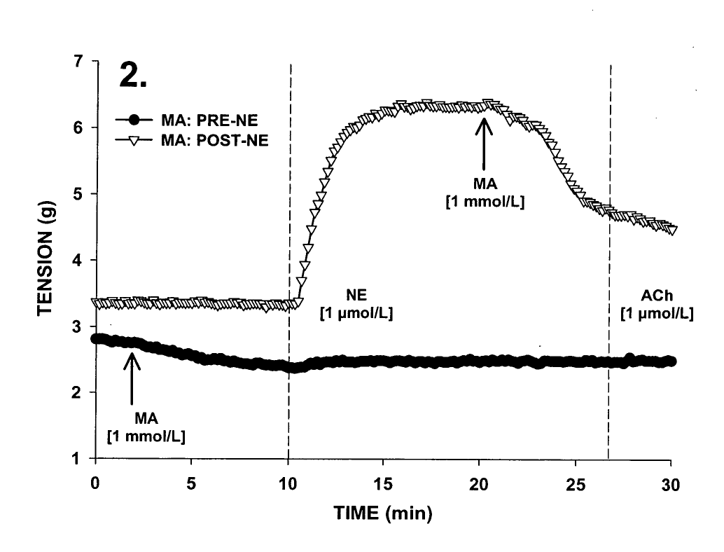

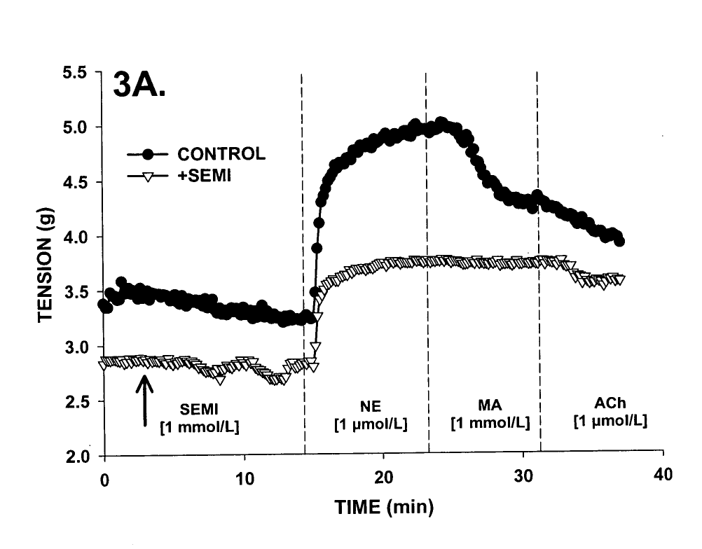

This Example includes data showing that SSAO induced relaxation is repeatable, reversible, and had no long-term inhibitory or toxic effects. Because the methylamine-induced relaxation expressed in all three vessels was especially prevalent in the LIMA, and because LIMA were the most frequently received blood vessel, methylamine relaxation was further studied in LIMA. Methylamine (MA) exposure (1 mmol/L; 10 min) inhibited or reduced NE contractions equally in unstimulated or NE-precontracted LIMA (mean+SE % reduction of NE-contraction: methylamine pre-NE, 53±7; methylamine post-NE, 61±5, n=5, 9 vessels, respectively; FIG. 2). The methylamine-induced relaxation in NE-precontracted LIMA was repeatable, reversible, and non-toxic. In NE-precontracted LIMA, a second methylamine relaxation was indistinguishable from the first, and a third methylamine relaxation also was elicited (mean±SE % relaxation: 1st relaxation, 72.0±7.5; 2nd relaxation=60.8±12.6; 3rd relaxation=82.1; n=5,5,1 vessels, respectively). Methylamine exposure had no observable long-lasting inhibitory or toxic effects in any vessel.

Semicarbazide Pretreatment Inhibited SSAO Substrate Mediated Relaxation in Isolated Human Blood Vessels

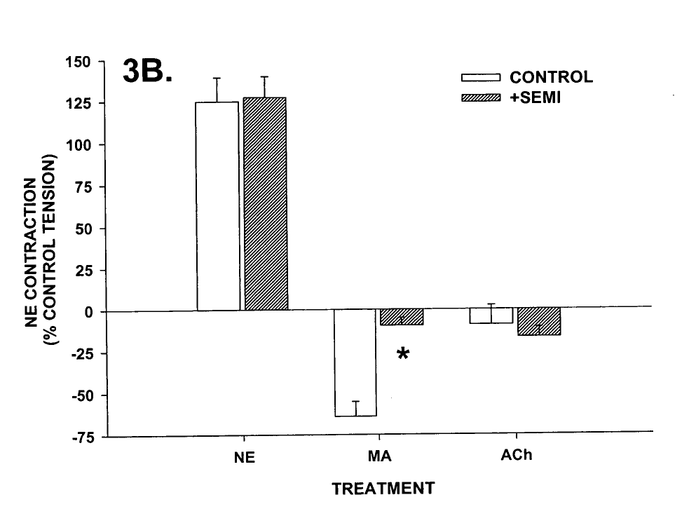

This Example includes data showing that vascular SSAO mediated relaxation caused by the introduction of SSAO substrates to blood vessels. The methylamine-induced relaxation in LIMA appeared dependent on vascular SSAO activity because semicarbazide pretreatment (1 mmol/L, 15 min) significantly reduced the methylamine-induced relaxation but had no effect on NE-induced contraction or ACh-induced relaxation (FIGS. 3A & 3B). Also, methylamine-induced relaxations in NE-precontracted LIMA were slowly reversed within 10 min of addition of 1 mmol/Lsemicarbazide (mean % reversal=63.0±15.8, n=2). Moreover, semicarbazide pretreatment appeared specific because the final vessel responses to HI K⁺, ACh, and SNP were not statistically different between paired LIMA vessels exposed to methylamine alone or semicarbazide plus methylamine (Table 3). Similarly, semicarbazide pretreatment (1 mmol/L, 15 min) completely blocked methylamine responses in RSV without effect on subsequent reactivity (data not shown; n=2).

TABLE 1
The relaxation (R) or contraction (C) responses in NE-precontracted
(1 μM) isolated human blood vessels, left internal mammary artery
(LIMA) and right saphenous vein (RSV), and rat thoracic aorta [TA;
18-20 week old Sprague-Dawley (Young SD) and 1-yr old Wistar
(Old W)] rats following 10 min exposure to 1 mmol/L methylamine,
formaldehyde, or hydrogen peroxide (H₂O₂).

| % Response | methylamine | | Formaldehyde | | H₂O₂ | |
|---|---|---|---|---|---|---|
| LIMA | | | | | | |
| C | 0.6 ± 0.3 | (30) | 3.7 ± 3.7 | (3) | 15.8 ± 5.3 | (9)* |
| R | 55.4 ± 3.9 | (30) | 37.3 ± 18.6 | (3) | 55.6 ± 9.0 | (9) |
| RSV | | | | | | |
| C | 17.4 ± 3.8 | (21) | 2.6 ± 2.6 | (3) | 25.4 ± 5.6 | (8) |
| R | 20.6 ± 4.3 | (21) | 31.7 ± 15.9 | (3) | 6.5 ± 3.2 | (8) |
| RAT | | | | | | |
| Young SD | | | | | | |
| Thoracic | | | | | | |
| Aorta | | | | | | |
| C | 12.5 ± 7.2 | (4) | 2.1 ± 2.1 | (4) | 41.2 ± 11.5 | (4)* |
| R | 8.3 ± 8.3 | (4) | 37.5 ± 21.6 | (4) | 50.8 ± 18.2 | (4) |
| Old W | | | | | | |
| C | 3.0 ± 3.0 | (3) | 2.6 ± 1.8 | (5) | 13.6 ± 3.1 | (6)* |
| R | 2.4 ± 2.4 | (3)* | 37.5 ± 16.2 | (5) | 56.6 ± 6.3 | (6)* |

Values are means ± SE in percentage change of NE-precontraction (1 μmol/L) tension.
(n) = number of vessels.
*= significant difference between value with asterisk and other toxicant values for same vessel and same response or significant difference only between values with asterisks for same vessel (P < 0.05).

TABLE 2
The apparent effective concentrations producing 50%
relaxation (R) or contraction (C) response (EC₅₀)
in isolated human blood vessels, left internal
mammary artery (LIMA), radial artery (RA), and
right saphenous vein (RSV), and rat thoracic
aorta (TA) to cumulative concentrations
of methylamine, formaldehyde, and hydrogen
peroxide (H₂O₂; 1-1000 μM).

| Apparent EC₅₀ | methylamine | | Formaldehyde | | H₂O₂ | |
|---|---|---|---|---|---|---|
| LIMA | | | | | | |
| C | not observed | | 315 | (1) | 175 ± 58 | (5) |
| R | 230 ± 30 | (8) | 315 ± 0 | (2) | 162 ± 41 | (9) |
| RA | | | | | | |
| C | 65 ± 48 | (3) | ND | | ND | |
| R | 280 ± 40 | (3) | ND | | ND | |
| RSV | | | | | | |
| C | 200 ± 52 | (6) | 315 | (1) | 220 ± 38 | (8) |
| R | 230 ± 28 | (5)* | 315 ± 0 | (2) | 315 ± 0 | (4)* |

Values are means ± SE in μmol/L interpolated from cumulative response curves of uncontracted and NE-precontracted blood vessels. Uncontracted vessel experimental data were not observably different from sensitivity data of contraction in NE-precontracted vessels and thus were pooled (where performed).
(n) = number of vessels.
ND = not determined.
*= significant difference between values with asterisks for same vessel (P < 0.05).

TABLE 3
Vascular responses were intact following methylamine exposure
(methylamine, 1 mM, 10 min) or methylamine plus sernicarbazide
exposure (SEMI+, 1 mM, 15 min) in isolated human left
internal mammary artery (LIMA, n = 7).

| | methylamine alone | SEMI + methylamine |
|---|---|---|
| HI K⁺ | 102 ± 23 | 127 ± 24 |
| ACh | 20 ± 11 | 16 ± 7 |
| SNP | 90 ± 11 | 83 ± 11 |
| High potassium contractions (HI K⁺, 100 mM), acetylcholine relaxations (ACh, 1 μM), and sodium nitroprusside relaxations (SNP, 100 μM) were performed in LIMA following prior exposure to methylamine or methylamine +SEMI and three bath changes with PSS. HI K⁺ contractions were calculated as a percentage of | | |
| # the control norepinephrine contraction; ACh and SNP relaxations were calculated as a percentage reduction in the HI K⁺ contraction. | | |

Values = means ± SE.
n = number of vessels.

TABLE 4
Vascular relaxations (%) and cGMP levels (pmol/mg protein)
following methylamine (MA, 1 mmol/L, 30-40 min) or sodium
nitroprusside (SNP, 100 μmol/L; 30-40 min) exposure in
NE-precontracted human left internal mammary artery (LIMA),
radial artery (RA), and right saphenous vein (RSV).

| | % Relaxation | [cGMP], pmol/mg |
|---|---|---|
| LIMA | | |
| Control (5) | — | 0.128 ± 0.034 |
| +MA, <50% relaxation (3) | 34.1 ± 7.1 | 0.067 ± 0.029 |
| +MA, >50% relaxation (6) | 87.8 ± 5.0 | 0.227 ± 0.066 |
| RSV | | |
| Control (5) | — | 0.118 ± 0.028 |
| +MA (5) | 57.2 ± 6.2 | 0.109 ± 0.030 |
| +SNP (6) | 100 ± 0 | 0.322 ± 0.065* |

Values = means ± SE.
Relaxations were calculated as a percentage reduction in the NE-precontraction tension.
(n) = number of vessels.
— indicates Control vessels were 5 matched sets of LIMA and RSV that were not subjected to NE-precontraction.
*indicates significantly greater cGMP level compared to control and methylamine-exposed RSV (P < 0.05).

The Role of Nitric Oxide, Prostanoids, and the Endothelium in the SSAO Substrate Mediated Relaxation in LIMA

This Example includes data showing that SSAO substrate induced relaxation can be distinguished from relaxation mediated by other mechanisms. The role of nitric oxide (NO) in the methylamine-induced relaxation in isolated human LIMA was investigated using ACh-induced relaxation as a marker. ACh-induced relaxations were most often present in the LIMA compared to RA or RSV (ACh addition usually produced small contraction in RSV; data not shown). The percentage methylamine-induced relaxation was vessel-specific and in general was associated with the ACh response, i.e., LIMA had the strongest ACh and methylamine relaxations, RSV had the strongest methylamine contractions and the weakest ACh and methylamine relaxations, and RA produced both strong methylamine contractions (=to RSV) and strong ACh and methylamine relaxations (=to LIMA; see FIG. 1A).

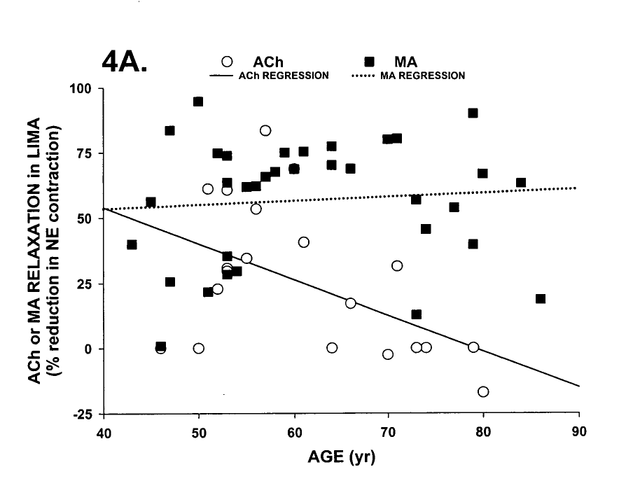

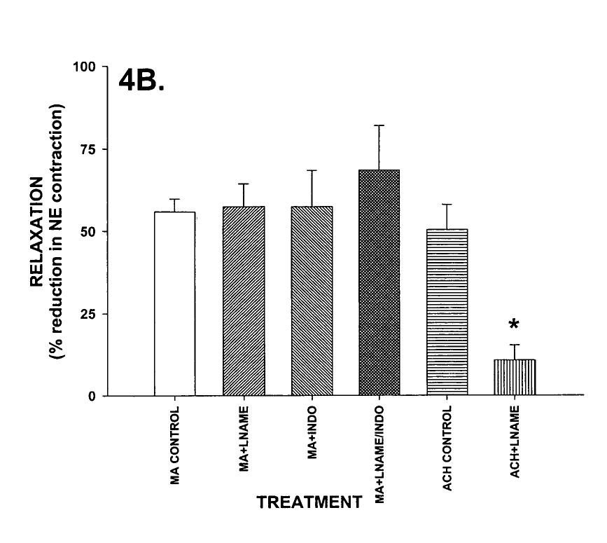

Despite the general positive association between ACh-induced relaxation and methylamine-induced relaxation observed across vessels, these two responses were not correlated within LIMA. In LIMA, the methylamine relaxation, on average, was >2 times stronger than the ACh-induced relaxation regardless of the order of addition (i.e., mean±SE in %; ACh added before or after MA; pre-MA ACh, 25±6; post-MA ACh, 14±6; n=21,20 vessels, respectively). More specifically, the percentage MA relaxation was not correlated with the percentage ACh relaxation or with patient age (a variable that was significantly correlated with the percentage ACh relaxation in LIMA; FIG. 4A). L-NAME pretreatment (200 μmol/L; 20 min) in a subset of LIMA rings possessing a relatively strong ACh relaxation significantly inhibited the ACh-induced relaxation but had no effect on MA-induced relaxation (FIG. 4C). In addition, neither INDO alone (100 μmol/L; 20 min) nor L-NAME+INDO pretreatment affected the MA-induced relaxation in NE-precontracted LIMA (FIG. 4C). Furthermore, the mean relaxation half time (t_{1/2}) for methylamine-induced relaxation was significantly longer in duration than the t_{1/2} for either ACh-induced relaxation in NE-precontracted LIMA or the SNP-induced relaxation in HI K⁺-precontracted LIMA (mean±SE in sec: MA, 139.9±35.8; ACh, 64.2±7.34; SNP, 78.3±6.6; n=9,9,8 vessels, respectively).

The role of hyperpolarization and H₂O₂ in relaxation was also investigated. Methylamine, formaldehyde, or H₂O₂ exposure (1 mmol/L; 10 min) in HI K⁺-precontracted LIMA typically resulted in small, sustained contractions (e.g., methylamine-treated: 6 of 9 responses were relatively small contractions, <10% increase in tension; H₂O₂-treated: 5 of 6 responses were <25% increase in tension). H₂O₂ produced significantly greater contractions than methylamine but not formaldehyde due to a few relatively large methylamine-stimulated relaxations (i.e., in 3 of 9 LIMA) while no relaxations were observed in any of the formaldehyde- or H₂O₂-exposed LIMA (mean±SE as % of HI K⁺ tension: MA, 1.7±3.9, n=9; formaldehyde, 12.9±2.4, n=3; H₂O₂, 21.1±7.7, n=9,3,6 vessels, respectively). Notably, 2 of the 3 methylamine relaxations, including the two strongest, were in LIMA from females. The subsequent relaxations of the HI K⁺-precontracted LIMA produced by ACh and SNP, respectively, were not significantly different between the methylamine-, formaldehyde-, and the H₂O₂-treated vessels (data not shown).

Role of cGMP in methylamine-induced Relaxation in Human LIMA. Since most vascular relaxations depend on cGMP production in vascular smooth muscle cells, the role of cGMP in methylamine-induced relaxation was investigated in LIMA and RSV. SNP (100 μmol/L) stimulated a statistically significant, 3-fold increase in cGMP levels (pmol/mg protein) in NE-precontracted RSV and the cGMP level was also increased in SNP-stimulated LIMA (% relaxation: 100; cGMP, 1.520; n=1) but not in RA (% relaxation: 73.9; cGMP, 0.099; n=1) (Table 4). However, 1 mmol/L methylamine exposure failed to significantly elevate cGMP in either NE-precontracted LIMA or RSV despite producing significant, long-lasting relaxations (Table 4). A strong positive correlation was observed in LIMA between the methylamine-induced % relaxation and the % B/Bo value (i.e., used to interpolate cGMP levels; r²=0.70, n=9) but a weaker relationship was present when the pmol cGMP levels were normalized to protein levels (r²=0.48, n=9; data not shown).

Human Blood Vessel SSAO Activity, Patient Age, and SSAO Inhibition

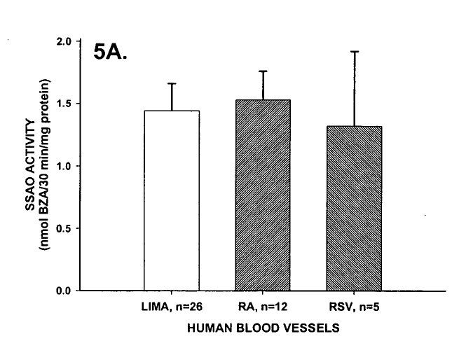

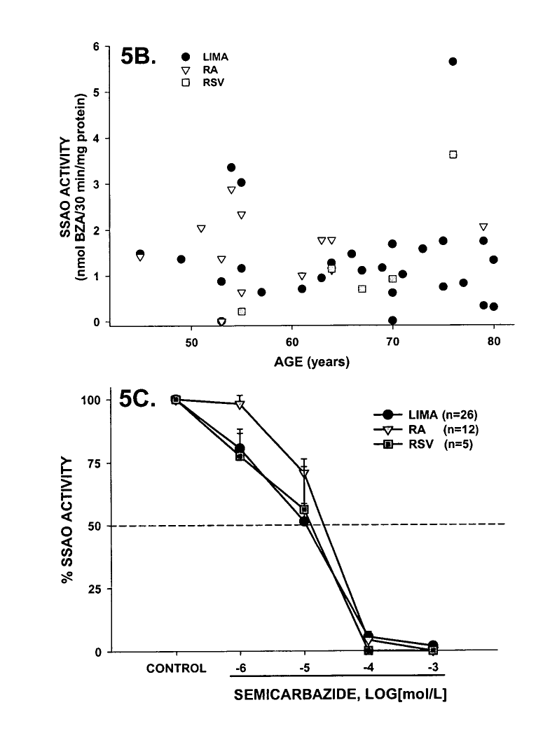

This Example includes data showing that SSAO substrates are expected to be effective for all patients regardless of age. Surprisingly, mean SSAO activity was similar among all three types of human blood vessels (FIG. 5A). For the age range examined, SSAO activity was not correlated with patient age for any vessel (FIG. 5B), but was significantly correlated between patient-matched LIMA and RSV (r2=0.76, p=0.054; n=5 vessels; data not shown), and similarly inhibited by semicarbazide (1, 10, 100, and 1,000 μmol/L) in all three blood vessel types (FIG. 5C; IC50s in μmol/L: LIMA, 16.7±19.5; RA, 21.2±11.2; RSV, 14.7±13.3; n=25,10,5 vessels, respectively).

SSAO Substrates for Blood Pressure Reduction Conventional wisdom indicates that chronic methylamine (MA) exposure induces vascular injury and promotes vascular disease, including atherosclerosis, in humans via semicarbazide-sensitive amine oxidase-mediated (SSAO) metabolism of methylamine to injurious metabolites: formaldehyde, hydrogen peroxide (H₂O₂), and ammonia (NH₃). But data reported herein show that methylamine induces long-lasting benign relaxation in isolated human blood vessels, and indicate that methylamine will be an effective vasorelaxant and/or hypotensive therapeutic compound. As discussed herein, data from an accidental human exposure to pure methylamine show that toxicological exposure to methylamine is lethal at high concentrations, but some of the cardiovascular symptoms presented following exposure support a pressure lowering mechanism of action. For example, low blood pressure and undetectable pulse were accompanied by tachycardia, high heart rate >100 bpm). Additionally, many patients presented with light to deep coma. Collectively these data suggest a potent systemic hypotension that reduced cerebral blood flow.

The direct effects of methylamine or other SSAO substrate exposure in the intact rodent or human cardiovascular system have not been measured. The performance of a suitable experiment, however, will provide additional evidence that SSAO substrates are effective for reducing blood pressure and/or treating hypotension. A suitable experiment is the administration of an SSAO substrate to an animal model, e.g., a rodent. Optimum dosages and routes of administration may be determined. Rodents are good models for determining the effects of methylamine because, like other mammals, they possess high levels of SSAO activity concentrated in the blood vessels (32). Also, rodents are less expensive and easier to maintain in the laboratory than larger mammals (e.g., primates, bovine, canine). More importantly, research has established that inhibition of SSAO leads to transient increases in methylamine excretion and blood pressure in rats (Conklin et al., unpublished; (34); Kumar et al., 1989). In addition, several transgenic and gene knockout mice are being used to probe the physiological/pathological role of cardiovascular SSAO activity. These mice could be useful for further development of this and related technologies.

Provided herein is an embodiment of such an experiment:

A. Route of Administration: Animals would be oral gavage fed methylamine in water. Controls would get water gavage.

B. Dosage: Two doses, one low [1 mg/kg body weight] and one high [100 mg/kg body weight], would be used. A low dose would be given on Day 1 and if no adverse effects are observed, the high dose would be given on Day 2. If a strong cardiovascular response is observed following low dose, then the dosage would be reduced for Day 2.

C. Variables Monitored: Cardiovascular variables (i.e., heart rate in bpm; systolic, mean, and diastolic blood pressure in mmHg) would be measured via a non-invasive blood pressure machine using the tail cuff method and a photoelectric sensor (NIBP; IITC, Inc., Woodland Hills, Calif.).

D. Data Collection: Signals from the NIBP will be recorded on a PC using IITC software at 5, 10, 20, 30, 60, 120, 300, and 1440 min following dosing.

The outcome of this experiment would be that a therapeutic dose of methylamine would stimulate systemic vasorelaxation and decrease mean arterial blood pressure. The onset may be fairly rapid (5-30 minutes to peak effect) but may be quite prolonged (hrs). The magnitude and duration of effects would likely be dose dependent. These effects may stimulate reflex tachycardia at some doses. Mortality is unlikely at these doses, although possible. The pretreatment of rats with the SSAO inhibitor, semicarbazide, would be expected to block all cardiovascular effects of methylamine. Other SSAO substrates and other compounds similar to methylamine, and compounds as described herein, are expected to have similar effects. Experimentation to optimize doses and routes of administration may be performed as needed.

Additional Aspects of the Examples

Without being bound to a particular hypothesized mechanism of action, it is clear that methylamine effects in isolated human blood vessels are dependent on vascular SSAO activity. Data from several experiments support this conclusion. The pretreatment of isolated LIMA with semicarbazide (1 mM; >15 min before addition of methylamine) led to significant inhibition of methylamine responses. The inhibition was specific for SSAO activity, since sernicarbazide pretreatment had no effects by itself and no effect on subsequent NE or HI K⁺ contractions or ACh relaxations. Moreover, semicarbazide pretreatment and post-treatment inhibition of methylamine relaxation in LIMA precludes a non-specific mechanism of action for methylamine (e.g., methylamine does not directly block adrenergic receptors; 31).

These data show, for the first time, that three human blood vessels used as coronary artery bypass graft vessels possess similar levels of relatively abundant, age-independent (age range=45-81 yrs) amine oxidase activity that is inhibited by semicarbazide in a concentration-dependent manner. These results are consistent with measurements made in other circumstances (14, 37; note that inhibition in human saphenous vein herein appears more sensitive; IC₅₀=500 vs 14.7 μmol/L; see 37, present study, respectively). Recently, similarly abundant SSAO activity was found in homogenized human coronary arteries of accident victims aged 7-71 yrs (14). Human aortic SSAO activity is approx. 10-times more concentrated than in human coronary arteries, LIMA, RA, RSV, and other human blood vessels but is also age-independent (23, 32). Similarly, the age of rats (12-14 weeks vs. 52 weeks) has little effect on thoracic aorta responses to methylamine exposure in our present study. Thus, age and perhaps atherosclerosis (23) appear to have little effect on SSAO expression in the blood vessel wall.

Dependence on SSAO activity is a hallmark of allylamine cardiovascular toxicity in vivo, in isolated blood vessels, and in cultured cardiovascular cells (2, 7, 50). The cellular toxicity of methylamine, in contrast, has a more complicated history since methylamine toxicity is orders of magnitude less toxic than allylamine in cultured cardiovascular cells (13, 30, 50). While allylamine and methylamine are relatively similar substrates for homogenized rat aortic SSAO activity (Kₘs=145.2 and 246.7 μmol/L, respectively; 47), human blood vessels have strikingly different Kₘs for methylamine (e.g., cerebral microvessels, 22 μM, 11; umbilical artery, 832 μM, see 33 for review). While the disparity in toxicity between these two amines is likely due to the metabolism of allylamine to acrolein and methylamine to the less toxic formaldehyde, it is likely that affinity and catalytic rates and, thus, specificity of various SSAOs for different amines vary dramatically from site to site (11, 33, 53). This has important implications for understanding the potential contribution of methylamine to differential cardiovascular pathology in humans (e.g., atherosclerosis, retinopathy, nephropathy, coronary artery disease; 53).

Without being bound to a particular theory of operation, it seems that SSAO mediates vasorelaxation by contributing to formation of H₂O₂ and formaldehyde. Since methylamine's effects in LIMA and RSV appear dependent on SSAO activity, it follows that methylamine's effects are likely due to one or more of methylamine's metabolites: formaldehyde, H₂O₂, and NH₃. The methylamine responses in LIMA and RSV are similar, qualitatively and quantitatively, to both formaldehyde- and H₂O₂-induced responses. For example, H₂O₂ (1 mmol/L) pretreatment inhibits subsequent NE contractions in rabbit aorta, while in precontracted vessels H₂O₂ induces contractions, relaxations, or biphasic responses dependent on the blood vessel type (25, 28, 40). More specifically, H₂O₂-induces biphasic responses in rat thoracic aorta and human RA and RSV are similar in appearance to our methylamine responses in isolated RA and RSV (28; present study). In the arteries, however, weak contractions are similar for methylamine and formaldehyde exposures whereas H₂O₂ is much more efficacious. Notably, vasospasm occurs more often in the human RA and the RSV compared with the LIMA, and our general reactivity findings are consistent with these data (10, 24). Additionally, methylamine, formaldehyde, and H₂O₂ effects generally are reversible at 1 mmol/L in rabbit aorta, rat thoracic aorta, and human blood vessels (25, 28, 40, present study). Finally, the apparent EC₅₀s for methylamine, formaldehyde, and H₂O₂ in all three human vessels are very similar (approximate range=200-300 μmol/L).

Methylamine-, formaldehyde-, and H₂O₂-induced relaxation in LIMA may be dependent on vascular smooth muscle cell (VSMC) membrane hyperpolarization via activation of K⁺ channels since HI K⁺-precontraction decreases their ability to stimulate relaxation. Hyperpolarization is used in H₂O₂-induced relaxation of rat aorta and porcine coronary artery (4, 28), and K⁺ channels may act as redox sensitive targets for H₂O₂ as proposed in the mechanism of hypoxic pulmonary vasoconstriction and hypoxia-induced vasodilation (see 1 for review). Moreover, H₂O₂ is considered an endothelial-derived hyperpolarizing factor (EDHF) in human mesenteric arteries but not in the carbachol-induced relaxation in human RA (21, 36). In addition, the relaxation (and generally low toxicity) observed with 1 mmol/L formaldehyde in human blood vessels, although surprising, is consistent with reports that formaldehyde (660 μmol/L) relaxes NE-precontracted but not 25 mmol/L KCl-depolarized rabbit aorta by inhibition of Ca⁺⁺ influx and NE inactivation in vitro (42).

Without being bound to a particular hypothesis, and regardless of which specific metabolite is involved, methylamine-induced relaxation appears independent of endothelial NO or prostanoid release in human LIMA. This conclusion is supported by the observation that inhibition of endothelial nitric oxide synthase activity with L-NAME significantly reduced the ACh relaxation but had no effect on the methylamine relaxation. Yet, if the endothelium were to be involved, it could perhaps be releasing an EDHF in response to methylamine metabolites. Hamilton et al., (2001) propose that blood vessels with reduced EDRF capacity compensate with enhanced EDHF production. Since no inverse relationship is observed, however, between ACh and methylamine relaxations in LIMAs from patients with significantly reduced EDRF, the more likely role of formaldehyde or H₂O₂ generated at the VSMC plasma membrane (i.e., the location of SSAO; 44) is that of autocrine and/or paracrine factor(s).

Without being bound to a particular hypothesis, it is unclear whether methylamine-induced relaxations are dependent on increased cGMP. While the methylamine-induced relaxation in RSV appears cGMP-independent, there is a weak positive association between cGMP levels and the % relaxation to methylamine in the LIMA. However, even though many relaxations are mediated by cGMP, it is possible that formation of formaldehyde and/or H₂O₂ at the VSMC plasma membrane directly relax(es) vessels by activation of K+ channels, thiol oxidation, inhibition of Ca++ influx, adrenergic inactivation, or some combination of mechanisms.

### References

1. Archer S and Michelakis E. The mechanism(s) of hypoxic pulmonary vasoconstriction: potassium channels, redox O₂ sensors, and controversies. NIPS 17:131-137, 2002.
2. Awasthi S and Boor P J. Semicarbazide protection from in vivo oxidant injury of vascular tissue by allylamine. Toxicol Letters 66:157-163, 1993.
3. Baba S, Watanabe Y, Gejyo F, and Arakawa M. High-performance liquid chromatographic determination of serum aliphatic amines in chronic renal failure. Clinica Chimica Acta 136:48-56, 1984.
4. Barlow R S and White R E. Hydrogen peroxide relaxes porcine coronary arteries by stimulating BK_{Ca} channel activity. Am J Physiol 275(44):H1283-H1289, 1998.
5. Boomsma F, Derkx F H, van den Meiracker A H, Man in't Veld A J, and Schalekamp M A. Plasma semicarbazide-sensitive amine oxidase activity is elevated in diabetes mellitus and correlates with glycosylated haemoglobin. Clin Sci 88(6):675-679, 1995.
6. Boomsma F, van den Meiracker A H, Winkel S, Aanstoot H J, Batstra M R, Man in't Veld A J, and Bruining G J. Circulating semicarbazide-sensitive amine oxidase is raised both in Type I (insulin-dependent), in Type II (non-insulin-dependent) diabetes mellitus and even in childhood Type I diabetes at first clinical diagnosis. Diabetologia 42:233-237, 1999.
7. Boor P J and Nelson T J. Allylamine cardiotoxicity: III. Protection by semicarbazide and in vivo derangements of monoamine oxidase. Toxicology 18:87-102, 1980.
8. Boor P J, Hysmith R M, and Sanduja R. A role for a new vascular enzyme in the metabolism of xenobiotic amines. Circ Res 66:249-252, 1990.
9. Boor P J, Trent M B, Lyles G A, Tao M, and Ansari G A S. Methylamine metabolism to formaldehyde by vascular semicarbazide-sensitive amine oxidase. Toxicology 73:251-258, 1992.
10. Cable D G, Caccitolo J A, Pfeifer E A, Daly R C, Dearani J A, Mullany C J, O'Brien T, Orszulak T A, and Schaff H V. Endothelial regulation of vascular contraction in radial and internal mammary arteries. Ann Thorac Surg 67:1083-1090, 1999.
11. Castillo V, Lizcano J M, and Unzeta M. Presence of SSAO in human and bovine meninges and microvessels. Neurobiol 7(3):263-272, 1999.
12. Conklin D J and Boor P J. Allylamine cardiovascular toxicity: evidence for aberrant vasoreactivity. Toxicol Appl Pharmacol 148:245-251, 1998.
13. Conklin D J, Langford S D, and Boor P J. Serum and cellular semicarbazide-sensitive amine oxidase in amine metabolism and cardiovascular toxicity. Toxicol Sci 46:386-392, 1998.
14. Conklin D J, Boyce C L, Trent M B, and Boor P J. Amine metabolism: A novel path to coronary artery vasospasm. Toxicol Appl Pharmacol 175:149-159, 2001.
15. Cracowski J-L, Stanke-Labesque F, Sessa C, Hunt M, Chavanon O, Devillier P, and Bessard G. Functional comparison of the human isolated femoral artery, internal mammary artery, gastroepiploic artery, and saphenous vein. Can J Physiol Pharmacol 77:770-776, 1999.
16. Dar M S, Morselli P L, and Bowman E R. The enzymatic systems involved in the mammalian metabolism of methylamine. Gen Pharmacol 16(6):557-560, 1985.
17. Ekblom J. Potential therapeutic value of drugs inhibiting semicarbazide-sensitive amine oxidase: vascular cytoprotection in diabetes mellitus. Pharmacol Res 37(2):87-92, 1998.
18. Enrique-Tarancon G, Marti L, Morin N, Lizcano J M, Unzeta M, Sevilla L, Camps M, Palacin M, Testar X, Carpene C, and Zorzano A. Role of semicarbazide-sensitive amine oxidase on glucose transport and GLUT4 recruitment to the cell surface in adipose cells. J Biol Chem 273(14):8025-8032, 1998.
19. Göktürk G, Garpenstrand H, Nilsson J, Nordquist J, Oreland L, and Forsberg-Nilsson K. Studies on semicarbazide-sensitive amine oxidase in patients with diabetes mellitus and in transgenic mice. Biochim et Biophys Acta 1647:88-91, 2001.
20. Gronvall J L E, Garpenstrand H, Oreland L, and Ekblom J. Autoradiographic imaging of formaldehyde adducts in mice: possible relevance for vascular damage in diabetes. Life Sci 63(9):759-768, 1998.
21. Hamilton C A, McPhaden A R, Berg G, Pathi V, and Dominiczak A F. Is hydrogen peroxide an EDHF in human radial arteries? Am J Physiol 280(6):H2451-H2455, 2001.
22. Hayes B E and Clarke D E. Semicarbazide-sensitive amine oxidase activity in streptozotocin diabetic rats. Res Comm Chem Path Pharmacol 69(1):71-83, 1990.
23. Hayes B E, Ostrow P T, and Clarke D E. Benzylamine oxidase in normal and atherosclerotic human aortae. Exper Mol Path 38:243-254, 1983.
24. He G-W. Arterial grafts for coronary artery bypass grafting: Biological characteristics, functional classification, and clinical choice. Ann Thorac Surg 67:277-284, 1999.
25. Iesaki T, Okada T, Yamaguchi H, and Ochi R. Inhibition of vasoactive amine induced contractions of vascular smooth muscle by hydrogen peroxide in rabbit aorta. Cardiovasc Res 28:963-968, 1994.
26. Jaakkola K, Kaunismaki K, Tohka S, Yegutkin G, Vanttinen E, Havia T, Pellinlemi L J, Virolainen M, Jalkanen S, and Salmi M. Human vascular adhesion protein-i in smooth muscle cells. Am J Pathol 155:1953-1965, 1999.
27. Kapeller-Adler R and Toda K. Uber das vorkommen von monomethylamine im harn. Biochem Zeischrift 248:403-425, 1932.
28. Karasu C. Increased activity of H2O2 in aorta isolated from chronically streptozotocin-diabetic rats: effects of antioxidant enzymes and enzyme inhibitors. Free Rad Biol Med 27(1/2):16-27, 1999.
29. Langford S D, Trent M B, Balakumaran A, and Boor P J. Developmental vasculotoxicity associated with inhibition of semicarbazide-sensitive amine oxidase. Toxicol Appl Pharmacol 155:237-244, 1999.
30. Langford, S D, Trent M B, and Boor P J. Cultured vascular smooth muscle cells are resistant to methylamine toxicity: no correlation to semicarbazide-sensitive amine oxidase. Cardiovasc Toxicol 1:51-60, 2001.
31. Lebrun P, Atwater I, Rosario L M, Herchuelz A, and Malaisse W J. Dissociation by methylamine of insulin release from glucose-induced electrical activity in isolated mouse islets of Langerhans. Metabolism 34:1122-1127, 1985.
32. Lewinsohn R, -Heinrich Bohm K, Glover V, and Sandler M. A benzylamine oxidase distinct from monoamine oxidase B—Widespread distribution in man and rat. Biochem Pharmacol 27:1857-1863, 1978.
33. Lyles G A. Mammalian plasma and tissue-bound semicarbazide-sensitive amine oxidase: Biochemical, pharmacological and toxicological aspects. Int J Biochem Cell Biol 28(3):259-274, 1996.
34. Lyles G A and McDougall S A. The enhanced daily excretion of urinary methylamine in rats treated with semicarbazide or hydralazine may be related to the inhibition of semicarbazide-sensitive amine oxidase activities. J Pharm Pharmacol 41:97-100, 1989.
35. Mafra I, Herbert P, Santos L, Barros P, and Alves A. Evaluation of biogenic amines in some Portuguese quality wines by HPLC fluorescence detection of OPA derivatives. Am J Enol Vitic 50(1):128-132, 1990.
36. Matoba T, Shimokawa H, Kubota H, Morikawa K, Fujiki T, Kunihiro I, Mukai Y, Hirakawa Y, and Takeshita A. Hydrogen peroxide is an endothelium-derived hyperpolarizing factor in human mesenteric arteries. Biochem Biophys Res Comm 290:909-913, 2002.
37. Meszaros Z, Csanyi A, Vallus G, Szombathy T, Karadi I, and Magyar K. Inhibitor sensitivity of human serum and vascular semicarbazide-sensitive amine oxidases. Neurobiology 8(2):215-223, 2000.
38. Mitchell S C and Zhang A Q. Methylamine in human urine. Clin Chim Acta 312:107-114, 2001.
39. Precious E, Gunn C E, and Lyles G A. Deamination of methylamine by semicarbazide-sensitive amine oxidase in human umbilical artery and rat aorta. Biochem Pharmacol 37(4):707-713, 1988.
40. Rodriguez-Martinez M A, Garcia-Cohen E C, Baena A B, Gonzalez R, Salaices M, and Marin J. Contractile responses elicited by hydrogen peroxide in aorta from normotensive and hypertensive rats. Endothelial modulation and mechanisms involved. Br J Pharmacol 125:1329-1335, 1998.
41. Salmi M, Yegutkin G G, Lehvonen R, Koskinen K, Salminen T, and Jalkanen S. A cell surface amine oxidase directly controls lymphocyte migration. Immunity 14:265-276, 2001.
42. Tani T. [Relaxation of vascular smooth muscle induced by formaldehyde (author's transl)]. Nippon Yakurigaku Zasshi 77(2):221-230, 1981.
43. Thornalley P J, McLellan A C, Lo T W C, Benn J, and Sonksen P H. Negative association between erythrocyte reduced glutathione concentration and diabetic complications. Clin Sci 91:575-582, 1996.
44. Wibo M, Duong A T, and Godfraind T. Subcellular location of semicarbazide-sensitive amine oxidase in rat aorta. Eur J Biochem 112:87-94, 1980.
45. Wingender W. High-performance liquid chromatographic method for the quantitative analysis of a synthetic copolymenr with antitumor activity (copovithane) and methylamine in human plasma and urine. Jrl Chromot 273:319-326, 1983.
46. Yang G-H, Wang Y M, Chen L, and Yin J-Y. Emergency treatment and care of 35 patients with monomethylamine poisoning. [in Chinese, English abstract]. Chung-Hua Hu Li Tsa Chih Chinese J Nurs 30(2):83-85, 1995.
47. Yu P. Oxidative deamination of aliphatic amines by rat aorta semicarbazide-sensitive amine oxidase. J Pharm Pharmacol 42:882-884, 1990.
48. Yu P H. Increase of formation of methylamine and formaldehyde in vivo after administration of nicotine and the potential cytotoxicity. Neurochem Res 23(9):1205-1210, 1998.
49. Yu P H and Dyck R F. Impairment of methylamine clearance in uremic patients and its nephropathological implications. Clin Nephro 49(5):299-302, 1998.
50. Yu P H and Zuo D-M. Oxidative deamination of methylamine by semicarbazide-sensitive amine oxidase leads to cytotoxic damage in endothelial cells. Possible consequences for diabetes. Diabetes 42:594-603, 1993.
51. Yu P H and Zuo D-M. Aminoguanidine inhibits semicarbazide-sensitive amine oxidase activity: implications for advanced glycation and diabetic complications. Diabetologia 40:1243-1250, 1997.
52. Yu P H, Lai C-T, and Zuo D-M. Formation of formaldehyde from adrenaline in vivo; a potential risk factor for stress-related angiopathy. Neurochem Res 22(5):615-620, 1997.
53. Yu P H, Wright S, Fan E H, Lun Z-R, Gubisne-Harberle D. Physiological and pathological implications of semicarbazide-sensitive amine oxidase. Biochim Biophys Acta 1647:193-199, 2003.
54. Conklin, D. J., Garney, M., Hall, K., Mueller, H., Trent, M., P. Boor. Poster Presentation at University of Wisconsin-Eau Claire Biology Department, 2001.
55. Conklin, D. J., Seminar at University of Wisconsin-Eau Claire Biology Department, April 2002.

1. A method of processing a blood vessel, the method comprising preparing a blood vessel for implantation in a patient and exposing the blood vessel to a physiologically acceptable solution that comprises an exogenous substrate for an SSAO enzyme.

2. The method of claim 1 wherein the exogenous substrate has a chemical formula of
R₁—X₁—NH₂
wherein R₁ is chosen from a group consisting of H, OH, NH₂, and COOH, and
wherein
(a) X₁ is an alkyl having between one and twelve carbons,
(b) X₁ is a C₆ aromatic ring, or
(c) X₁ comprises a single C₆ aromatic ring and further comprises between one and eleven alkyl carbons.

3. The method of claim 2 wherein X₁ is CH₂.

4. The method of claim 3 wherein the exogenous substrate is present in the physiological solution at a concentration of between 0.01 and 100 millimolar.

5. The method of claim 3 wherein X₁ is CH₂, and R₁ is H, whereby the substrate has the formula CH₃NH₂.

6. The method of claim 2 wherein X₁ comprises a C₆ aromatic ring.

7. The method of claim 6 wherein R₁ is H.

8. The method of claim 7 wherein the exogenous substrate is present in the solution at a concentration of between 0.01 and 100 millimolar.

9. The method of claim 1 wherein the exogenous substrate has a chemical formula of 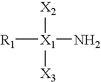
wherein R₁ is chosen from a group consisting of H, OH, NH₂, and COOH, X₂ is chosen from a group consisting of H, OH, NH₂, COOH, and alkyls having between one and three carbons, X₃ is chosen from a group consisting of H, OH, NH₂, COOH, and alkyls having between one and three carbons, and
wherein
(a) X₁ is an alkyl having between one and twelve carbons,
(b) X₁ is a C₆ aromatic ring, or
(c) X₁ comprises a single C₆ aromatic ring and further comprises between one and eleven alkyl carbons.

10. The method of claim 9 wherein the exogenous substrate is present in the solution at a concentration of between 0.01 and 100 millimolar.

11. The method of claim 9 wherein the exogenous substrate is present in the solution at a concentration of between 0.1 and 10 millimolar.

12. The method of claim 1 wherein the physiological solution comprises a buffer having an Osmolarity in the range of about 280 to about 350 milliOsmolar that buffers the solution to maintain a pH in a range of about 7.0 to about 7.8.

13. A composition comprising an in vitro blood vessel, a physiologically acceptable solution that comprises an exogenous buffer that provides a physiological pH, and a concentration of an exogenous substrate for an SSAO enzyme, wherein the concentration of the exogenous substrate is at least great enough to relax the blood vessel exposed to the solution.

14. The composition of claim 13 wherein the exogenous substrate has a chemical formula of
R₁—X₁—NH₂
wherein R₁ is chosen from a group consisting of H, OH, NH₂, and COOH, and
wherein
(a) X₁ is an alkyl having between one and twelve carbons,
(b) X₁ is a C₆ aromatic ring, or
(c) X₁ comprises a single C₆ aromatic ring and further comprises between one and eleven alkyl carbons.

15. The composition of claim 14 wherein X₁ is CH₂.

16. The composition of claim 14 wherein the exogenous substrate is present in the physiological solution at a concentration of between 0.01 and 100 millimolar.

17. The composition of claim 14 wherein X₁ is CH₂, and R₁ is H, whereby the substrate has the formula CH₃NH₂.

18. The composition of claim 14 wherein X₁ comprises a C₆ aromatic ring.

19. The composition of claim 18 wherein R₁ is H.

20. The composition of claim 19 wherein the exogenous substrate is present in the solution at a concentration of between 0.01 and 100 millimolar.

21. The composition of 14 wherein the exogenous substrate is present in the solution at a concentration of between 0.01 and 100 millimolar.

22. The composition of claim 14 wherein the exogenous substrate has a chemical formula of 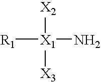
wherein R₁ is chosen from a group consisting of H, OH, NH₂, and COOH, X₂ is chosen from a group consisting of H, OH, NH₂, COOH, and alkyls having between one and three carbons, X₃ is chosen from a group consisting H, OH, NH₂, COOH, and alkyls having between one and three carbons, and
wherein
(a) X₁ is an alkyl having between one and twelve carbons,
(b) X₁ is a C₆ aromatic ring, or
(c) X₁ comprises a single C₆ aromatic ring and further comprises between one and eleven alkyl Carbons.

23. A medicament comprising a purified exogenous substrate for an SSAO enzyme and a pharmaceutical carrier.

24. A method of using a medicament, the method comprising administering the medicament of claim 23 to a patient.

25. The medicament of claim 23 wherein the exogenous substrate has a chemical formula of
R₁—X₁—NH₂
wherein R₁ a member of a group consisting of H, OH, NH₂, and COOH, and
wherein
(a) X₁ is an alkyl having between one and twelve carbons,
(b) X₁ is a C₆ aromatic ring, or
(c) X₁ comprises a single C₆ aromatic ring and further comprises between one and eleven alkyl carbons.

26. A method of using a medicament, the method comprising administering the medicament of claim 25 to a patient.

27. The medicament of claim 25 wherein X₁ is CH₂.

28. The medicament of claim 25 wherein X₁ is CH₂, and R₁ is H, whereby the substrate has the formula CH₃NH₂.

29. The medicament of claim 25 wherein X₁ comprises a C₆ aromatic ring.

30. The medicament of claim 29 wherein R₁ is H.

31. The medicament of claim 29 comprising between 1 and 10,000 milligrams of the exogenous substrate.

32. The medicament of claim 23 comprising between 1 and 10,000 milligrams of the exogenous substrate.

33. The medicament of claim 23 wherein the exogenous substrate has a chemical formula of 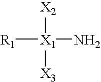
wherein R1 is a member of a group consisting of H, OH, NH₂, and COOH, X₂ is a member of the group consisting of H, OH, NH₂, COOH, and alkyls having between one and three carbons, X₃ is a member of the group consisting of H, OH, NH₂, COOH, and alkyls having between one and three carbons, and
wherein
(a) X₁ is an alkyl having between one and twelve Carbons,
(b) X₁ is a C₆ aromatic ring, or
(c) X₁ comprises a single C₆ aromatic ring and further comprises between one and eleven alkyl Carbons.

34. The medicament of claim 33 comprising between 1 and 10,000 milligrams of the exogenous substrate.

35. A method of using the medicament of claim 33, the method comprising administering the medicament to a patient.

36. The medicament of claim 23 wherein the medicament comprises a member of a group consisting of a pill, a granule, tablet, capsule, suspension, suppository, pessary, lotion, solution, cream, ointment, dusting powder, powder, paste, foam, aerosol, mist, /atomizing solution, surgical glue, medical tape, and patch.

37. The medicament of claim 23 wherein the carrier comprises a member of a group consisting of a starch, cellulose, malt, gelatin, talc, oil, glycol, polyol, ester, agar, pharmaceutically-acceptable salt, pharmaceutically-acceptable acid, and pharmaceutically-acceptable base.

38. A method of using the medicament of claim 23, the method comprising administering the medicament to a patient.

39. A kit for treating a patient, the kit comprising the medicament of claim 23 and instructions for use of the medicament.

40. The kit of claim 39 wherein the instructions are a member of the group of instructions consisting of written, electronic, web-interactive, email, label, brochure, slide, and handout.

41. A method of treating a patient for high blood pressure, the method comprising administering to the patient a medicament comprising a purified exogenous substrate for an SSAO enzyme and a pharmaceutical carrier to thereby lower the blood pressure of the patient.

42. The method of claim 41 wherein the exogenous substrate has a chemical formula of
R₁—X₁—NH₂
wherein R₁ a member of a group consisting of H, OH, NH₂, and COOH, and
wherein
(a) X₁ is an alkyl having between one and twelve carbons,
(b) X₁ is a C₆ aromatic ring, or
(c) X₁ comprises a single C₆ aromatic ring and further comprises between one and eleven alkyl carbons.

43. The method of claim 42 wherein X₁ is CH2.

44. The method of claim 42 wherein X₁ is CH₂, and R₁ is H, whereby the substrate has the formula CH₃NH₂.

45. The method of claim 42 wherein X₁ comprises a C₆ aromatic ring.

46. The method of claim 42 wherein R₁ is H.

47. The method of claim 42 wherein the patient receives between 10-10,000 mg/kg of the exogenous substrate.

48. The method of claim 42 wherein the patient receives between 10-1,000 mg/kg of the exogenous substrate.

49. The method of claim 42 wherein the exogenous substrate has a chemical formula of 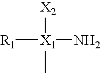
wherein R1 is a member of a group consisting of H, OH, NH₂, and COOH, X₂ is a member of the group consisting of H, OH, NH₂, COOH, and alkyls having between one and three carbons, X₃ is a member of the group consisting of H, OH, NH₂, COOH, and alkyls having between one and three carbons, and
wherein
(a) X₁ is an alkyl having between one and twelve Carbons,
(b) X₁ is a C₆ aromatic ring, or
(c) X₁ comprises a single C₆ aromatic ring and further comprises between one and eleven alkyl Carbons.

50. The method of claim 49 wherein the patient receives between 10-10,000 mg/kg of the exogenous substrate.

51. The method of claim 49 wherein the patient receives between 10-1,000 mg/kg of the exogenous substrate.

52. The method of claim 41 wherein the medicament comprises a member of a group consisting of a pill, a granule, tablet, capsule, suspension, suppository, pessary, lotion, solution, cream, ointment, dusting powder, powder, paste, foam, aerosol, mist, /atomizing solution, surgical glue, medical tape, and patch.

53. The method of claim 41 wherein the carrier comprises a member of a group consisting of a starch, cellulose, malt, gelatin, talc, oil, glycol, polyol, ester, agar, pharmaceutically-acceptable salt, pharmaceutically-acceptable acid, and pharmaceutically-acceptable base.

54. A kit for treating a patient, the kit comprising instructions for use of a medicament in treating a patient for high blood pressure according to the method of claim 41.

55. The kit of claim 54 wherein the instructions are a member of the group of instructions consisting of written, electronic, web-interactive, email, label, brochure, slide, and handout.
